(19) United States
(12) Patent Application Publication
(10) Pub. No.: US 2025/0091895 A1
(43) Pub. Date: Mar. 20, 2025

(54) INDIUM OXIDE NANORODS, METHODS FOR PREPARING AND USING SAME

(71) Applicant: YANGTZE DELTA REGION INSTITUTE (HUZHOU), UNIVERSITY OF ELECTRONIC SCIENCE AND TECHNOLOGY OF CHINA, Huzhou City (CN)

(72) Inventors: Zhaojun QIN, Huzhou City (CN); Weiwu CHEN, Huzhou City (CN)

(21) Appl. No.: 18/487,608

(22) Filed: Oct. 16, 2023

(30) Foreign Application Priority Data
Sep. 15, 2023 (CN) 2023111973973

(51) Int. Cl. C01G 15/00; B82Y 15/00; B82Y 40/00; G01N 33/00

(52) U.S. Cl. CPC C01G 15/00; G01N 33/0027; B82Y 15/00; B82Y 40/00; C01P 2002/01; C01P 2004/16

(57) ABSTRACT

Provided herein is a method for preparing indium oxide nanorods, comprising the following steps: S1: placing indium oxide powder at a central temperature control area of a tube furnace, and then placing a cleaned silicon wafer downstream from the indium oxide powder; S2: evacuating inside the tube furnace, and then continuing to introduce argon gas; and S3: adjusting a program to heat up the central temperature control area of the tube furnace and maintaining it at a temperature, and then naturally cooling to obtain a indium oxide nanorods grown on the surface of the silicon wafer.

### CROSS-REFERENCE TO RELATED APPLICATIONS

This application claims priority under 35 U.S.C. § 119(a) to Chinese Patent Application No. 2023111973973, filed on Sep. 15, 2023, which is hereby incorporated by reference herein in its entirety.

### TECHNICAL FIELD

The present disclosure belongs to the field of toxic and harmful gas sensing and detection, and specifically relates to indium oxide nanorods, methods for preparing the indium oxide nanorods and a use of the indium oxide nanorods in formaldehyde gas detection.

### BACKGROUND ART

As a typical one-dimensional nanomaterial, nanorods have obvious advantages in terms of size and structure and can usually exhibit good electrical properties. Therefore, the development of nanorod materials can help solve the dilemma faced by optoelectronic materials that cannot be used due to size limitations. It has good applications in making some small and micro electronic devices, such as micro emitters, gas sensors, diodes, etc., which is of great application value.

Indium oxide is a semiconductor material with a bandgap width of 2.9 eV. It has been proven that indium oxide can exhibit good chemical stability under most conventional conditions and has also been proven to be suitable for gas detection. Formaldehyde gas, which people are often exposed to due to daily factors such as decoration and furniture, is highly harmful to people's bodies and is of greater concern among various toxic and harmful gases.

For a gas sensor, an important indicator is the response recovery time. According to the article “Research Progress of Metal Oxide Semiconductor Sensors for Rapid Detection of Formaldehyde in the Field” in the journal Shandong Chemical Industry, statistics have been collected on most of the current indium oxide formaldehyde gas sensors. It can be seen that the response recovery time of the current indium oxide formaldehyde gas sensors is generally longer than thirty or even forty seconds. On the other hand, an important indicator of gas sensitive materials is operating temperature required for the material to achieve gas-sensing response. For indium oxide materials, the operating temperature is typically around 150° C., posing problems such as a prolonged response recovery time and a relatively high operating temperature.

Therefore, developing a simple preparation method of indium oxide nanorods, which exhibit high sensitivity and rapid response to formaldehyde gas, can provide a good improvement for the development of the application of formaldehyde gas detectors.

### SUMMARY

Bearing in mind the deficiencies in the prior art, the present disclosure provides indium oxide nanorods, methods for preparing the indium oxide nanorods and a use of the indium oxide nanorods in formaldehyde gas detection. The present disclosure simplifies the preparation method of indium oxide nanorods, meanwhile, the obtained indium oxide nanorods exhibit good sensitivity to formaldehyde gas.

To the accomplishment of the foregoing, the technical solutions adopted by the present disclosure are as follow:

A method for preparing indium oxide nanorods, comprising the following steps:
S1: placing indium oxide powder at a central temperature control area of a tube furnace, and then placing a cleaned silicon wafer downstream from the indium oxide powder;
S2: evacuating inside the tube furnace, and then continuing to introduce argon gas; and
S3: adjusting a program to heat up the central temperature control area of the tube furnace and maintaining it at a temperature, and then naturally cooling to obtain indium oxide nanorods grown on surface of the silicon wafer.

According to one or more embodiments, the indium oxide powder in step S1 has a purity of 99.99%.

According to one or more embodiments, the cleaned silicon wafer is placed at a position of 10-15 cm downstream from the indium oxide powder in step S1.

According to one or more embodiments, inside the tube furnace is evacuated to 20-50 Pa in step S2.

According to one or more embodiments, the argon gas is introduced at a flow rate of 25-30 sccm in step S2.

According to one or more embodiments, the argon gas has a purity of 99.9%.

According to one or more embodiments, the program is adjusted to raise the temperature of the central temperature control area of the tube furnace to 1050-1100° C. and maintaining it for 110-120 minutes in step S3.

The present disclosure also provides a use of indium oxide nanorods in formaldehyde gas detection, comprising:
(1) scraping the indium oxide nanorods into ethanol to obtain a suspension and dripping the resulting suspension onto a new silicon wafer; and
(2) using a tungsten wire to evenly wrap the surface of the silicon wafer obtained in step (1), depositing a metal film on the surface of the silicon wafer attached by indium oxide nanorods wrapped with the tungsten wire, and removing the tungsten wire to obtain the indium oxide nanorod formaldehyde gas sensor for formaldehyde gas detection.

According to one or more embodiments, in step (2), the tungsten wire has a diameter of 15-20 μm, the metal film has a thickness of 50-60 nm, and the metal film material is selected from any one of titanium, gold, and copper.

The beneficial effects of the present disclosure are as below:
(1) the equipment and raw materials required for the present disclosure are inexpensive and the preparation process is simple;
(2) the preparation method of the present disclosure can obtain indium oxide nanorods, the size of which is very uniform. And as the nanorods have a great specific surface area, the exposure to formaldehyde gas is greatly increased, with a single indium oxide nanorod alone being highly responsive to formaldehyde gas. With the fast adsorption and desorption rates obtained from a large contact surface, the response recovery time is only about 15 seconds. It begins to exhibit a noticeable response to formaldehyde gas from 80° C., and reaches its optimal state at 160° C.; and
(3) at present, the response recovery time of indium oxide formaldehyde gas sensors is generally greater than thirty or even forty seconds. On the other hand, an important indicator of gas sensitive materials is operating temperature required for the material to achieve gas-sensing response. For indium oxide materials, the operating temperature is usually around 150° C. With good electrical transmission properties and a large specific surface area, nanorods effectively promote the direct recharge between the nanorods and formaldehyde gas during the gas-sensing response process, such that the indium oxide nanorods prepared by the present disclosure can reduce the response recovery time to about fifteen seconds and have a good gas-sensing response intensity at reduced operating temperatures from 80-160° C., which has significant technical advantages.

### BRIEF DESCRIPTION OF THE DRAWINGS

FIG. 1 is a transmission electron microscope image of the indium oxide nanorods according to one or more embodiments of the present disclosure.

FIG. 2 is a photo of a single indium oxide nanorod formaldehyde gas sensor according to one or more embodiments of the present disclosure.

FIG. 3 is a gas sensitivity diagram of an indium oxide nanorod formaldehyde gas sensor according to one or more embodiments of the present disclosure.

FIG. 4 is the temperature performance plot of a single indium oxide nanorod formaldehyde gas sensor according to one or more embodiments of the present disclosure.

### DETAILED DESCRIPTION

The technical solutions in the embodiments of the present disclosure will be clearly and completely described below with reference to the accompanying drawings according to one or more embodiments of the present disclosure. Obviously, the described embodiments are only some of the embodiments of the present disclosure. Based on the embodiments of the present disclosure, all other embodiments obtained by those of ordinary skill in the art without creative efforts fall within the scope of protection of the present disclosure.

### Example 1

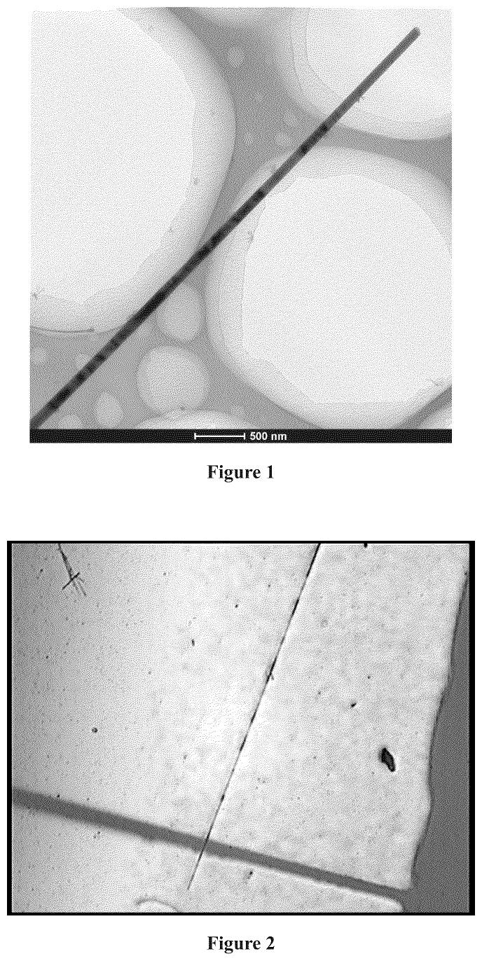

A method for preparing indium oxide nanorod formaldehyde gas sensor, comprising the following steps:
S1: placing indium oxide powder with a content of 99.99% at a central temperature control area of a tube furnace, and then placing a cleaned silicon wafer at a position of 15 cm from the indium oxide powder;
S2: evacuating inside the tube furnace to 30 Pa, and then continuing to introduce argon gas with a purity of 99.9% at a flow rate of 30 sccm;
S3: adjusting a program to heat up the central temperature control area of the tube furnace to 1100° C. and maintaining it for 120 min, and then naturally cooling to obtain indium oxide nanorods grown on the surface of the silicon wafer (see FIG. 1 for the transmission electron microscope image);
S4: scraping the uniformly sized indium oxide nanorods into ethanol to obtain a suspension and dripping the resulting suspension onto a new silicon wafer; and
S5: using a tungsten wire with a diameter of 15 μm to evenly wrap the surface of the silicon wafer obtained in step S4, depositing a metal film with a thickness of 60 nm on the surface of the silicon wafer attached by indium oxide nanorods wrapped with tungsten wire, and removing the tungsten wire to obtain a single indium oxide nanorod formaldehyde gas sensor (see FIG. 2).

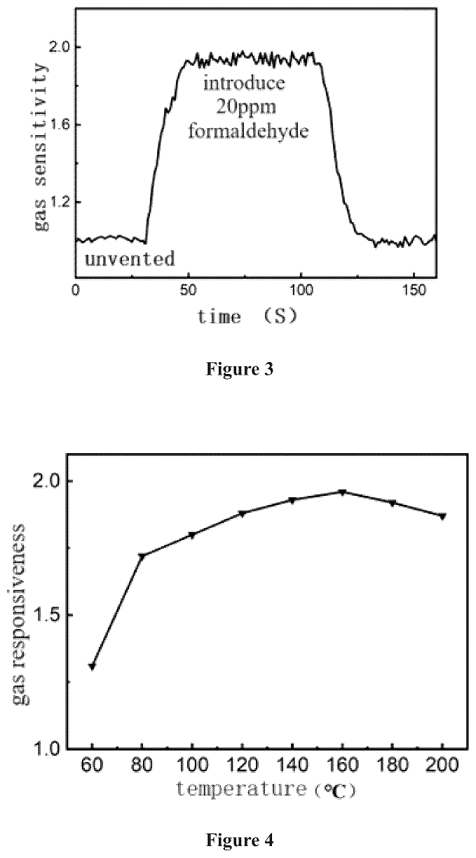

The single indium oxide nanorod formaldehyde gas sensor prepared in this example is connected to a current detection system and the sensor is placed in a gas environment containing 20 ppm concentration of formaldehyde. It can be seen that just a single indium oxide nanorod can generate obvious current intensity changes in formaldehyde gas (see FIG. 3), and its response recovery time is 15 seconds. The temperature of the sensor is controlled by a heating stage (see FIG. 4). It is found that at 160° C., the sensor exhibits a response amplitude of 1.96 times to formaldehyde gas with a concentration of 20 ppm, and even at 80° C., the sensor still exhibits 1.72 times gas sensitivity response strength, which shows a large temperature detection range.

### Example 2

A method for preparing indium oxide nanorod formaldehyde gas sensor, comprising the following steps:
S1: placing indium oxide powder with a content of 99.99% at a central temperature control area of a tube furnace, and then placing a cleaned silicon wafer at a position of 10 cm from the indium oxide powder;
S2: evacuating inside the tube furnace to 22 Pa, and then continuing to introduce argon gas with a purity of 99.9% at a flow rate of 25 sccm;
S3: adjusting a program to heat up the central temperature control area of the tube furnace to 1100° C. and maintaining it for 120 min, and then naturally cooling to obtain indium oxide nanorods grown on the surface of the silicon wafer;
S4: scraping the uniformly sized indium oxide nanorods into ethanol to obtain a suspension and dripping the resulting suspension onto a new silicon wafer; and
S5: using a tungsten wire with a diameter of 15 μm to evenly wrap the surface of the silicon wafer obtained in step S4, depositing a metal film with a thickness of 51 nm on the surface of the silicon wafer attached by indium oxide nanorods wrapped with the tungsten wire, and removing the tungsten wire to obtain a single indium oxide nanorod formaldehyde gas sensor.

### Example 3

A method for preparing indium oxide nanorod formaldehyde gas sensor, comprising the following steps:
S1: placing indium oxide powder with a content of 99.99% at a central temperature control area of a tube furnace, and then placing a cleaned silicon wafer at a position of 12 cm from the indium oxide powder;
S2: evacuating inside the tube furnace to 25 Pa, and then continuing to introduce argon gas with a purity of 99.9% at a flow rate of 25 sccm;
S3: adjusting a program to heat up the central temperature control area of the tube furnace to 1100° C. and maintaining it for 120 min, and then naturally cooling to obtain indium oxide nanorods grown on the surface of the silicon wafer;
S4: scraping the uniformly sized indium oxide nanorods into ethanol to obtain a suspension and dripping the resulting suspension onto a new silicon wafer; and
S5: using a tungsten wire with a diameter of 16 μm to evenly wrap the surface of the silicon wafer obtained in step S4, depositing a metal film with a thickness of 53 nm on the surface of the silicon wafer attached by indium oxide nanorods wrapped with the tungsten wire, and removing the tungsten wire to obtain single indium oxide nanorod formaldehyde gas sensor.

### Example 4

A method for preparing indium oxide nanorod formaldehyde gas sensor, comprising the following steps:
S1: placing indium oxide powder with a content of 99.99% at a central temperature control area of a tube furnace, and then placing a cleaned silicon wafer at a position of 13 cm from the indium oxide powder;
S2: evacuating inside the tube furnace to 28 Pa, and then continuing to introduce argon gas with a purity of 99.9% at a flow rate of 26 sccm;
S3: adjusting a program to heat up the central temperature control area of the tube furnace to 1100° C. and maintaining it for 120 min, and then naturally cooling to obtain indium oxide nanorods grown on the surface of the silicon wafer;
S4: scraping the uniformly sized indium oxide nanorods into ethanol to obtain a suspension and dripping the resulting suspension onto a new silicon wafer; and
S5: using a tungsten wire with a diameter of 17 μm to evenly wrap the surface of the silicon wafer obtained in step S4, depositing a metal film with a thickness of 54 nm on the surface of the silicon wafer attached by indium oxide nanorods wrapped with the tungsten wire, and removing the tungsten wire to obtain single indium oxide nanorod formaldehyde gas sensor.

### Example 5

A method for preparing indium oxide nanorod formaldehyde gas sensor, comprising the following steps:
S1: placing indium oxide powder with a content of 99.99% at a central temperature control area of a tube furnace, and then placing a cleaned silicon wafer at a position of 12 cm from the indium oxide powder;
S2: evacuating inside the tube furnace to 40 Pa, and then continuing to introduce argon gas with a purity of 99.9% at a flow rate of 27 sccm;
S3: adjusting a program to heat up the central temperature control area of the tube furnace to 1100° C. and maintaining it for 120 min, and then naturally cooling to obtain indium oxide nanorods grown on the surface of the silicon wafer;
S4: scraping the uniformly sized indium oxide nanorods into ethanol to obtain a suspension and dripping the resulting suspension onto a new silicon wafer; and
S5: using a tungsten wire with a diameter of 20 μm to evenly wrap the surface of the silicon wafer obtained in step S4, depositing a metal film with a thickness of 58 nm on the surface of the silicon wafer attached by indium oxide nanorods wrapped with the tungsten wire, and removing the tungsten wire to obtain single indium oxide nanorod formaldehyde gas sensor.

### Example 6

A method for preparing indium oxide nanorod formaldehyde gas sensor, comprising the following steps:
S1: placing indium oxide powder with a content of 99.99% at a central temperature control area of a tube furnace, and then placing a cleaned silicon wafer at a position of 15 cm from the indium oxide powder;
S2: evacuating inside the tube furnace to 29 Pa, and then continuing to introduce argon gas with a purity of 99.9% at a flow rate of 30 sccm;
S3: adjusting a program to heat up the central temperature control area of the tube furnace to 1100° C. and maintaining it for 120 min, and then naturally cooling to obtain indium oxide nanorods grown on the surface of the silicon wafer;
S4: scraping the uniformly sized indium oxide nanorods into ethanol to obtain a suspension and dripping the resulting suspension onto a new silicon wafer; and
S5: using a tungsten wire with a diameter of 16 μm to evenly wrap the surface of the silicon wafer obtained in step S4, depositing a metal film with a thickness of 60 nm on the surface of the silicon wafer attached by indium oxide nanorods wrapped with the tungsten wire, and removing the tungsten wire to obtain single indium oxide nanorod formaldehyde gas sensor.

### Example 7

A method for preparing indium oxide nanorod formaldehyde gas sensor, comprising the following steps:
S1: placing indium oxide powder with a content of 99.99% at a central temperature control area of a tube furnace, and then placing a cleaned silicon wafer at a position of 12 cm from the indium oxide powder;
S2: evacuating inside the tube furnace to 35 Pa, and then continuing to introduce argon gas with a purity of 99.9% at a flow rate of 30 sccm;
S3: adjusting a program to heat up the central temperature control area of the tube furnace to 1100° C. and maintaining it for 120 min, and then naturally cooling to obtain indium oxide nanorods grown on the surface of the silicon wafer;
S4: scraping the uniformly sized indium oxide nanorods into ethanol to obtain a suspension and dripping the resulting suspension onto a new silicon wafer; and
S5: using a tungsten wire with a diameter of 18 μm to evenly wrap the surface of the silicon wafer obtained in step S4, depositing a metal film with a thickness of 56 nm on the surface of the silicon wafer attached by indium oxide nanorods wrapped with the tungsten wire, and removing the tungsten wire to obtain single indium oxide nanorod formaldehyde gas sensor.

### Example 8

A method for preparing indium oxide nanorod formaldehyde gas sensor, comprising the following steps:
S1: placing indium oxide powder with a content of 99.99% at a central temperature control area of a tube furnace, and then placing a cleaned silicon wafer at a position of 15 cm from the indium oxide powder;
S2: evacuating inside the tube furnace to 45 Pa, and then continuing to introduce argon gas with a purity of 99.9% at a flow rate of 28 sccm;
S3: adjusting a program to heat up the central temperature control area of the tube furnace to 1100° C. and maintaining it for 120 min, and then naturally cooling to obtain indium oxide nanorods grown on surface of the silicon wafer;
S4: scraping the uniformly sized indium oxide nanorods into ethanol to obtain a suspension and dripping the resulting suspension onto a new silicon wafer; and
S5: using a tungsten wire with a diameter of 16 μm to evenly wrap the surface of the silicon wafer obtained in step S4, depositing a metal film with a thickness of 55 nm on the surface of the silicon wafer attached by indium oxide nanorods wrapped with the tungsten wire, and removing the tungsten wire to obtain single indium oxide nanorod formaldehyde gas sensor.

The above merely describes specific embodiments of the present disclosure, which is not intended to limit the scope of protection of the present disclosure. Any modifications, equivalent variations or substitutions, and improvements made within the spirit and principle of the present disclosure by those skilled in the art according to the disclosed technical scope should be included in the protection scope of the present disclosure.

What is claimed is:

1. A method for preparing indium oxide nanorods, comprising the following steps:
S1: placing indium oxide powder at a central temperature control area of a tube furnace, and then placing a cleaned silicon wafer downstream from the indium oxide powder;
S2: evacuating inside the tube furnace, and then continuing to introduce argon gas; and
S3: adjusting a program to heat up the central temperature control area of the tube furnace and maintaining it at a temperature, and then naturally cooling to obtain indium oxide nanorods grown on the surface of the silicon wafer.

2. The method for preparing indium oxide nanorods according to claim 1, wherein the indium oxide powder has a purity of 99.99% in step S1.

3. The method for preparing indium oxide nanorods according to claim 1, wherein the cleaned silicon wafer is placed at a position of 10-15 cm downstream from the indium oxide powder in step S1.

4. The method for preparing indium oxide nanorods according to claim 1, wherein the inside of the tube furnace is evacuated to 20-50 Pa in step S2.

5. The method for preparing indium oxide nanorods according to claim 1, wherein the argon gas is introduced at a flow rate of 25-30 sccm in step S2.

6. The method for preparing indium oxide nanorods according to claim 5, wherein the argon gas has a purity of 99.9%.

7. The method for preparing indium oxide nanorods according to claim 1, wherein the program is adjusted to raise the temperature of the central temperature control area of the tube furnace to 1050-1100° C. and maintaining it for 110-120 minutes in step S3.

8. Indium oxide nanorods prepared according to the method of claim 1.

9. Use of indium oxide nanorods for detecting formaldehyde gas according to claim 8, comprising the following steps:
(1) scraping the indium oxide nanorods into ethanol to obtain a suspension and dripping the resulting suspension onto a new silicon wafer; and
(2) using a tungsten wire to evenly wrap the surface of the silicon wafer obtained in step (1), depositing a metal film on the surface of the silicon wafer attached by indium oxide nanorods wrapped with the tungsten wire, and removing the tungsten wire to obtain the indium oxide nanorod formaldehyde gas sensor for formaldehyde gas detection.

10. Use of indium oxide nanorods for detecting formaldehyde gas according to claim 9, wherein in step (2), the tungsten wire has a diameter of 15-20 μm, the metal film has a thickness of 50-60 nm, and the metal film material is selected from any one of titanium, gold, and copper.
